(19) United States
(12) Patent Application Publication
(10) Pub. No.: US 2019/0304570 A1
(43) Pub. Date: Oct. 3, 2019

(54) MDS TO AML TRANSITION AND PREDICTION METHODS THEREFOR

(71) Applicant: NANTOMICS, LLC, Culver City, CA (US)

(72) Inventors: Stephen Charles Benz, Culver City, CA (US); Andrew Nguyen, Culver City, CA (US); Andrew J. Sedgewick, Culver City, CA (US); Christopher Szeto, Culver City, CA (US)

(21) Appl. No.: 16/345,686

(22) PCT Filed: Oct. 27, 2017
(86) PCT No.: PCT/US2017/058793

§ 371 Date: Apr. 26, 2019

Related U.S. Application Data
(60) Provisional application No. 62/413,917, filed on Oct. 27, 2016.
(60) Provisional application No. 62/429,036, filed on Dec. 1, 2016.

(51) Int. Cl. G16B 25/00; C12Q 1/6886; G16B 5/00

(52) U.S. Cl. CPC G16B 25/00; C12Q 2600/156; G16B 5/00; C12Q 1/6886

(57) ABSTRACT

Contemplated systems and methods allow for prediction of time for MDS to AML transition using a predictive model that is based on selected features with significant differential expression levels and/or pathway activity between MDS to AML cells.

This application claims priority to U.S. provisional applications with the Ser. No. 62/413,917, filed Oct. 27, 2016, and 62/429,036, filed Dec. 1, 2016.

### FIELD OF THE INVENTION

The field of the invention is method of omics analysis for prediction and analysis of MDS (myelodysplastic syndrome) to AML (acute myeloid leukemia) progression.

### BACKGROUND

The following description includes information that may be useful in understanding the present invention. It is not an admission that any of the information provided herein is prior art or relevant to the presently claimed invention, or that any publication specifically or implicitly referenced is prior art.

All publications and patent applications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Myelodysplastic syndrome (MDS) constitutes a group of clonal hematopoietic disorders characterized by bone marrow failure, dysplasia, and an increased likelihood of progression to acute myeloid leukemia (AML). MDS is generally classified as “primary” (or de novo) and “treatment-related” (secondary to prior cytotoxic chemotherapy) and both are thought to arise due to abnormalities in hematopoietic stem cell self-renewal and differentiation.

Many different conditions are grouped together under the “MDS” umbrella based on common clinical characteristics, thus accounting for the wide heterogeneity observed. Diagnosis of patients with this disease can be difficult at times. Similarly, the assigning of prognosis and the selection of appropriate therapy require careful application of prognostic scoring systems taking into account clinical characteristics (e.g., cytopenias, age, performance status) and cytological parameters (e.g., blast count, morphology, karyotype). Factors such as poor cytogenetics are associated with decreased survival in MDS.

Several factors have been identified that can significantly impact the prognosis and selection of therapy for MDS patients, such as cytogenetics, patient performance status, and red blood cell (RBC) transfusion dependence. Numerous studies have shown that patient performance status is inversely associated with overall or event-free survival in patients receiving intensive chemotherapy for MDS or AML, particularly in older individuals. Appropriate diagnosis and classification of MDS depends on accurate assessments of both clinical features and laboratory/pathology findings (e.g., blast count, peripheral blood counts, cytogenetics). To this end, well-prepared bone marrow smears and biopsy specimens are essential. Unfortunately, such methods require significant time and review by trained professionals, adding significant cost.

More recently, various genetic conditions have been associated with treatment sensitivity, prognosis, survival time, etc. for MDS and AML. For example, patients with del(5q) MDS who failed to achieve sustained erythroid or cytogenetic remission after treatment with lenalidomide were shown to have an increased risk for clonal evolution and AML progression (see Ann Hematol. 2010 April; 89(4):365-74). In another study, the Wilms' tumor gene WT1 was reported to be a good marker for diagnosis of disease progression of myelodysplastic syndromes (see Leukemia 1999 March; 13(3):393-9), and a combined assessment of WT1 and BAALC gene expression at diagnosis was reported to possibly improve leukemia-free survival prediction in patients with myelodysplastic syndromes (see Leuk Res. 2015 August; 39(8):866-73). Similarly, individual mutations in the TET2 gene were reported to be diagnostic markers for MDS or AML as discussed in WO2010/087702.

In still further known tests, somatic, non-silent mutational signatures were reported to predict survivability of MDS as is discussed in US 2014/0127690, and WO 2013/056184 teaches methods for testing whether a drug, compound, diet, therapy or treatment is effective or efficacious for preventing, ameliorating, slowing the progress of, stopping or slowing the metastasis of, or for causing a full or partial remission of, a cancer, or a cancer stem cell, or a leukemia cancer stem cell. However, none of the known methods allows for a robust prediction of time of progression from MDS to AML.

Therefore, there is still a need for improved prognostic tests that can predict the time of progression from MDS to AML, which helps guide physicians in the selection of appropriate treatment options for patients diagnosed with MDS.

### SUMMARY OF THE INVENTION

The inventive subject is directed to various methods in which the time for progression of MDS to AML can be predicted based on certain omics features, especially by using differentially expressed genes and/or inferred pathway activities in a regression-based model.

In one aspect of the inventive subject matter, the inventors contemplate a method of predicting time of progression from MDS to AML that includes a step of quantifying expression of a plurality of genes of a sample containing myelodysplastic cells, wherein the plurality of genes have an above-average difference between MDS and AML with respect to at least one of mRNA expression and inferred pathway activity. In another step, the plurality of genes having the above-average difference between MDS and AML is used in a prediction model to calculate a likely time of progression from MDS to AML.

While in some embodiments, the plurality of genes have an above-average difference between MDS and AML with respect to mRNA expression, in other embodiments the plurality of genes have an above-average difference between MDS and AML with respect to inferred pathway activity. It is further contemplated that the plurality of genes are selected from the group consisting of CHD4, GPATCH2L, FAM212A, EXT2, MACF1, RTKN, ZSCAN2, RNF220, YEATS2, ERGIC1, ZNF618, MBTD1, CXXC5, and DUSP10. Viewed from a different perspective, the prediction model may be based on a plurality of differentially expressed genes in which at least 50 genes are differentially expressed as determined by t-test and an alpha of 0.05 (as for example shown in FIG. 7).

While not limiting to the inventive subject matter, the prediction model may be built using a regression algorithm, and more preferably a lasso least-angle regression algorithm. It is further preferred that the prediction model provides predictions up to at least 120 months, and/or that the step of quantifying expression of the plurality of genes uses whole transcriptome RNAseq data. Moreover, it is contemplated that contemplated methods may further include a step of identifying a druggable target in the whole transcriptome RNAseq data, and optionally a step of generating or updating a report with a treatment recommendation.

Therefore, in yet another aspect of the inventive subject matter, the inventors also contemplate a method of generating a model for predicting time for MDS to AML transition. Preferred models will generally include a step of quantifying expression of a plurality of genes of a sample containing MDS cells, and another step of quantifying expression of a plurality of genes of a sample containing AML cells (typically performed using whole transcriptome RNAseq data). Optionally, inferred pathway activities are then calculated for the plurality of genes of the sample containing MDS cells and the plurality of genes of the sample containing AML cells. In yet another step, a plurality of genes are identified with an above-average difference between the MDS cells and the AML cells with respect to at least one of mRNA expression and inferred pathway activity, and the plurality of genes with the above-average difference between the MDS cells and the AML cells are used to build a prediction model that calculates a likely time of progression from MDS to AML.

Most typically, the plurality of genes have an above-average difference between MDS and AML with respect to mRNA expression and/or an above-average difference between MDS and AML with respect to inferred pathway activity. As noted above, it is contemplated that the prediction model may be based on a plurality of differentially expressed genes in which at least 50 genes are differentially expressed as determined by t-test and an alpha of 0.05. For example, suitable genes with above-average difference between the MDS cells and the AML cells include CHD4, GPATCH2L, FAM212A, EXT2, MACF1, RTKN, ZSCAN2, RNF220, YEATS2, ERGIC1, ZNF618, MBTD1, CXXC5, and DUSP10. In further contemplated aspects, the prediction model is built using a regression algorithm (e.g., lasso least-angle regression algorithm).

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a graph depicting mutational burden as a function of transition time from MDS to AML.

FIG. 2 is a graph depicting clonal and sub-clonal fraction of neoepitopes in tumors of AML patients.

FIG. 3 is a graph depicting changes in expression of all genes in AML cells relative to gene expression in MDS.

FIG. 4 is a graph depicting changes in expression of selected genes in AML cells relative to gene expression in MDS.

FIG. 5 is one graph depicting changes in inferred pathway activity of selected genes in AML cells relative to gene expression in MDS.

FIG. 6 is another graph depicting changes in inferred pathway activity of selected genes in AML cells relative to gene expression in MDS.

FIG. 7 is a heat map of significant differentially expressed genes between MDS and AML cells of the same patient.

FIG. 8A is a graph depicting a time-to-progression function, and FIG. 8B is a table listing genes used in the function and performance parameters for the function.

### DETAILED DESCRIPTION

The inventors have now discovered that the time for progression of MDS to AML can be predicted with relatively high accuracy using a predictive algorithm that is built on differentially expressed genes and/or genes with differential pathway activity. Notably, differential expression and/or differential pathway activity of selected genes held significantly stronger predictive power than overall mutation rates, single gene mutations, and presence or type of neoepitopes generated by mutations in MDS in the progression to AML. The inventors also discovered that while the coding clonal mutational burden in MDS was relatively low, there was a pervasive significant change in overall gene expression (with the exception of CD34) as the disease moved from MDS to AML.

With respect to specific mutations in selected genes, the inventors also discovered a small subset of mutations that may be associated (causally or indirectly) with the progression of MDS to AML. Specifically, and as is shown in more detail below, most AML cells exhibited a higher expression in Myc, FLT3 (which also sowed higher expression in Myb), and APF2. On the other hand, transcription decreased substantial downregulation of FOXM1 as the disease progressed and a reduced expression of GATA1.

Thus, on the basis of these observations, various manners or predicting progression, and especially time of progression of MDS to AML are contemplated using these observations. In most preferred aspects, prediction will not simply be predicated on the quantification of a single marker as variability with a single marker would be unlikely to provide a graduated prediction (e.g., within a time resolution of 3 months, 2 months, or 1 month, or 2 weeks, or even 1 week). Therefore, the inventors investigated whether a multi-factorial analysis using most differentially expressed genes and/or pathway activities could be used to produce a prediction model that can provide information on the likely time required for a patient to progress from MDS to AML. Such graduated information is especially important for choice of an appropriate treatment. In addition, a multi-factorial predictive algorithm is also advantageous as MDS is a collection of various sub-diseases for which individual diagnostic and prognostic makers are difficult to identify.

Based on the unexpected discovery that many genes had a negative expression bias upon transition from MDS to AML, the inventors investigated whether or not there was a differential expression pattern to one or more genes. Notably, and as shown in more detail below, genes with significant differential expression between MDS and AML served as statistically meaningful features in machine learning in an analysis that correlated time to progress from MDS to AML with expression values of these genes. As a consequence, a statistical model could be defined that allowed prediction of MDS to AML progression in a quantitative manner (as opposed to simply diagnosing a state of MDS or AML). Surprisingly, and as also shown in more detail below, the resultant model was relatively simple and required only relatively low numbers of expression data of selected genes.

### Example

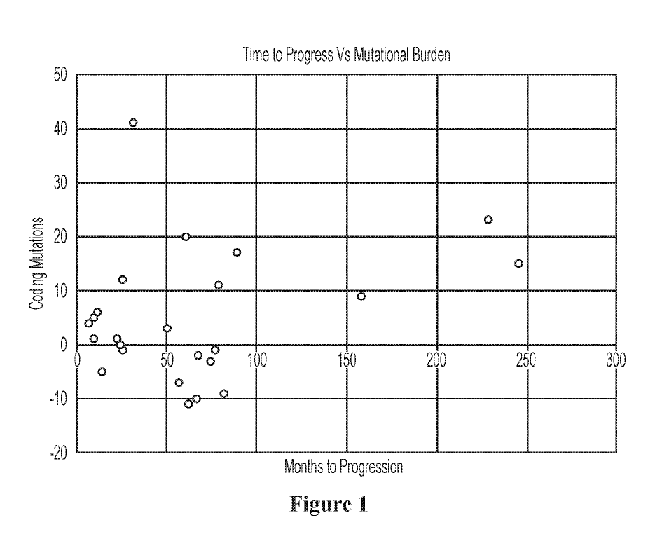

In a first attempt to identify a predictive marker of progression of MDS to AML, the inventors compared patient data with different times of progression and mutational burden, and particularly mutational burden of genetic sequences that encode proteins. Omics analysis was performed using whole genome sequencing of MDS and AML cells from the same patient, and incremental location guided synchronous alignment using BAMBAM, as for example described in U.S. Pat. No. 9,721,062. FIG. 1 depicts an exemplary result from such analysis. As is readily apparent, in a patient population with a progression time of less than 38 months, the median mutational change was at about +2.5 coding mutations, while in a patient population with a progression time of more than 38 months and less than 80 months the median mutational change was at about −2.0 coding mutations. On the other hand, in patients with a progression time of more than 80 months, the median mutational change was at about +15.0 coding mutations. While such increase was at least seemingly significant, the data failed to provide a reliable foundation for a quantitative and predictive model.

When analyzing the mutational changes for all genes as a possible guide for predicting transition time of MDS to AML, the inventors noted that several genes had a significant differential mutational burden. Interestingly, some genes lost mutations in the progression of MDS to AML, while other genes gained mutations as is exemplarily shown in Table 1. Notably, several patients had FLT3 and IDH1 mutations. Moreover, it was noted that large genes such as NBPF genes were more affected, possibly due to mutations by chance. Therefore, these mutations appear to represent passenger mutations rather than driver mutations. While significant in terms of specificity, these mutational changes were not sufficient for a quantitative predictive model. Most notably, the shutting down of a great number of genes at AML stage would be consistent with a situation where a blast population emerges where the cells complete two milestones: They do not differentiate and do not apoptose. Thus, those specific genes and pathways are deemed to have significance for diagnostic and prognostic use. For example, genes associated with viability like BCL2 family and those associated with apoptosis like CASPASE pathway or pro-inflammatory cytokine cascade. Involvement of Ribosomal proteins and their dosage effect of haplo-insufficiency rather than genetic mutations has been established in MDS and also found in congenital anemias. Ribosomal issues link congenital and acquired anemias.

TABLE 1

| Gene | MDS | AML | Diff |
|---|---|---|---|
| NBPF20 | 26 | 20 | −6 |
| ZNF844 | 5 | 0 | −5 |
| MUC17 | 10 | 7 | −3 |
| ATXN2 | 3 | 0 | −3 |
| TUBGCP | 3 | 0 | −3 |
| MED16 | 4 | 1 | −3 |
| CCNB3 | 6 | 3 | −3 |
| CACNA1 | 5 | 2 | −3 |
| SYN2 | 5 | 2 | −3 |
| MAGEC1 | 4 | 1 | −3 |
| DUSP13 | 3 | 0 | −3 |
| MUC20 | 4 | 1 | −3 |
| SETD1B | 4 | 1 | −3 |
| FCGBP | 5 | 2 | −3 |
| MAPK3 | 4 | 1 | −3 |
| DPY19L2 | 3 | 0 | −3 |
| NBPF8 | 3 | 11 | 8 |
| RUNX1 | 5 | 11 | 6 |
| ZBTB42 | 1 | 7 | 6 |
| MUC19 | 2 | 7 | 5 |
| RBMXL3 | 3 | 8 | 5 |
| MUC4 | 1 | 5 | 4 |
| CNTNAP | 0 | 4 | 4 |
| WASH1 | 2 | 6 | 4 |
| FLT3 | 0 | 3 | 3 |
| IDH1 | 1 | 4 | 3 |
| KIAA075 | 0 | 3 | 3 |
| MUC5B | 2 | 5 | 3 |

Analysis Limited to Mutations with >10% AF

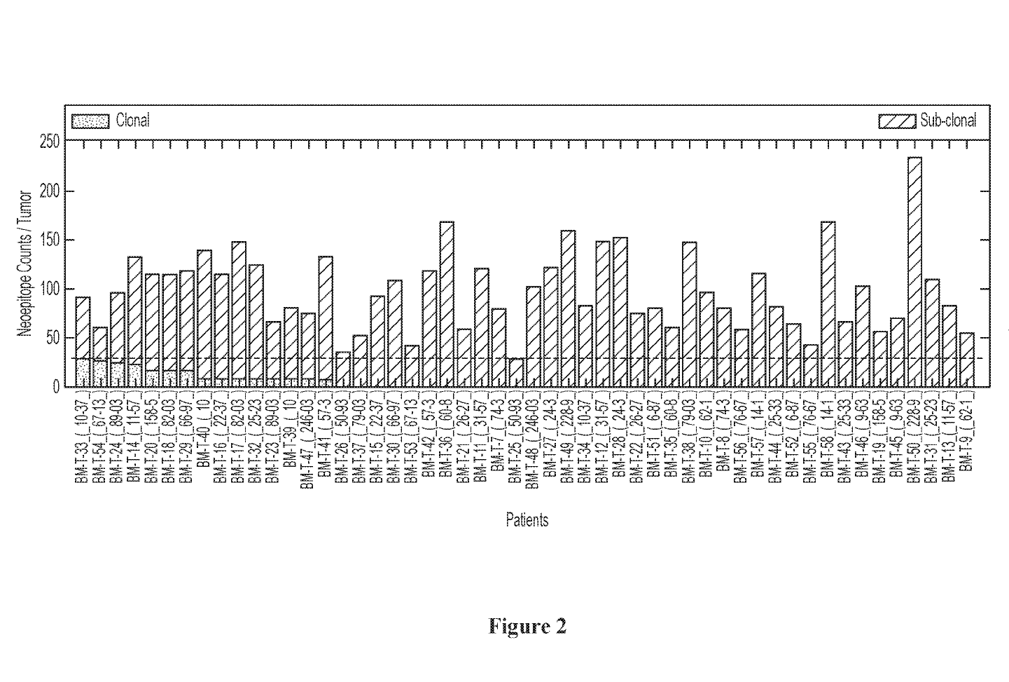

Using the same comparative whole genome analysis and further considering expression of the mutated sequences, the inventors further investigated whether or not neoepitopes in coding and expressed DNA segments could serve as a basis for a quantitative predictive model, and exemplary results are shown in FIG. 2 where each bar represents a differential record (MDS versus AML) for an individual patient. Darker portions in each bar of the graph indicate clonal neoepitopes (clonal fraction of neoepitopes at least 90%), while the lighter portions represent sub-clonal neoepitopes (clonal fraction of neoepitopes less than 90%). As it turned out, neither clonal nor sub-clonal neoepitopes could serve as basis for a quantitative predictive model.

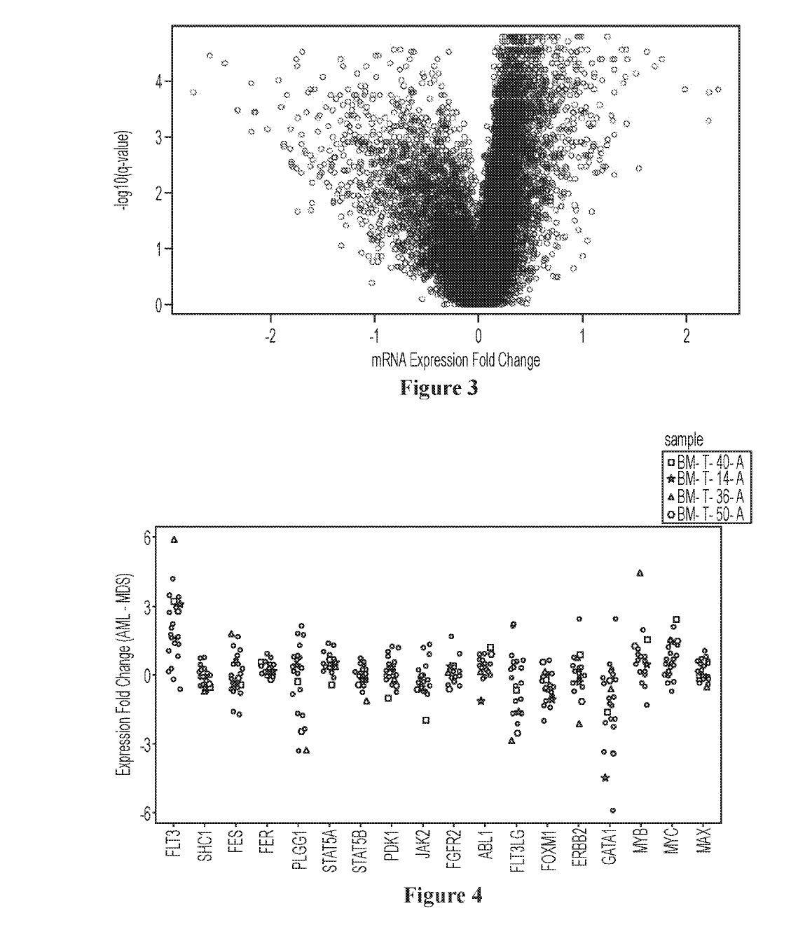

Surprisingly, however, the inventors observed upon analysis of gene expression that a substantial portion of genes were expressed to a significantly lower degree as can be seen in the graph of FIG. 3. Here, each data point depicted as a circle represents the expression strength differential for a single gene (as n-fold mRNA) plotted against the −log₁₀ FDR adjusted p-value (q-value) for the data point. As can be readily seen from the graph, while a notable fraction of genes were expressed at substantially the same rate, several genes were strongly overexpressed while many other genes were significantly under-expressed upon transition from MDS to AML. Thus, in a first approximation, it is contemplated that the overall expression level of genes could serve as a basis for calculating the transition time from MDS to AML. While generating a quantitative and predictive model from a large quantity of RNAseq data (e.g., at least 100 genes, at least 500 genes, at least 1,000 genes, at least 5,000) is not excluded, the inventors considered that selected genes may be candidate features of a quantitative and predictive model that can use few data points at a desired predictive accuracy.

To that end, the inventors investigated on the basis of RNAseq data (and in some cases also whole genome or exome sequencing data) which of the differentially expressed genes had significant and strong difference in expression. Moreover, the inventors also used the function of the differentially expressed genes in a pathway analysis algorithm to identify those expressed genes that produced the largest difference in inferred pathway activity. More specifically, the inventors determined the effect of the differentially expressed genes using a pathway recognition algorithm using data integration on genetic models as is described in WO 2013/062505. Of course, it should be appreciated that numerous alternative pathway analysis models are also deemed suitable, and all known pathway analysis models are contemplated herein.

More specifically, Table 2 lists the genes with the largest median paired differences of mRNA expression (AML versus MDS), while Table 3 lists the genes with the largest median paired differences of inferred pathway activity (AML versus MDS). Table 4 lists the genes with the largest median inferred pathway activity (AML normalized to paired MDS).

TABLE 2

| Gene Name | Statistic | Median Difference | p. value | q. value |
|---|---|---|---|---|
| CRISP3 | 335 | −2.74 | 5.04E−06 | 1.57E−04 |
| CAMP | 346 | −2.58 | 2.98E−07 | 3.49E−05 |
| LCN2 | 344 | −2.43 | 5.66E−07 | 4.74E−05 |
| DEFA1B | 329 | −2.31 | 1.60E−05 | 3.27E−04 |
| DEFA1 | 329 | −2.31 | 1.60E−05 | 3.27E−04 |
| BAALC | 15 | 2.30 | 4.08E−06 | 1.39E−04 |
| CD34 | 16 | 2.21 | 5.04E−06 | 1.57E−04 |
| NPR3 | 26 | 2.21 | 3.19E−05 | 5.10E−04 |
| LTF | 338 | −2.18 | 2.62E−06 | 1.08E−04 |
| HBM | 321 | −2.18 | 8.03E−05 | 8.00E−04 |
| PGLYRP1 | 328 | −2.15 | 1.91E−05 | 3.62E−04 |
| DEFA3 | 328 | −2.14 | 1.91E−05 | 3.62E−04 |
| DEFA4 | 322 | −2.08 | 5.16E−05 | 7.10E−04 |
| SHANK3 | 15 | 1.98 | 4.08E−06 | 1.39E−04 |
| OLFM4 | 339 | −1.92 | 2.09E−06 | 9.55E−05 |
| MMP8 | 330 | −1.89 | 1.33E−05 | 2.92E−04 |
| TRIM10 | 316 | −1.87 | 1.26E−04 | 1.33E−03 |
| HBD | 315 | −1.87 | 1.45E−04 | 1.46E−03 |
| PLBD1 | 334 | −1.84 | 6.17E−06 | 1.76E−04 |
| EPB42 | 314 | −1.81 | 1.66E−04 | 1.61E−03 |

TABLE 3

| Gene Name | Statistic | Median | p. value | q. value |
|---|---|---|---|---|
| MYC/Max (complex) | 34.0 | 4.171 | 1.09E−04 | 0.01334 |
| ATF2 | 34.0 | 2.184 | 1.09E−04 | 0.01334 |
| GATA1 | 313.0 | −1.575 | 1.90E−04 | 0.01480 |
| SMARCC1 | 18.0 | 1.448 | 7.54E−06 | 0.00841 |
| ATF2_(dimer)_(complex) | 47.0 | 1.382 | 5.91E−04 | 0.02248 |
| ATF2/JUND_(complex) | 24.0 | 1.195 | 2.27E−05 | 0.01198 |
| DUSP10 | 23.0 | 1.052 | 1.91E−05 | 0.01064 |
| POLR3D | 7.0 | 1.050 | 7.20E−05 | 0.01227 |
| ATF2/TIP49B_(complex) | 50.0 | 1.002 | 8.35E−04 | 0.02563 |
| MBOAT2 | 272.0 | −0.988 | 4.76E−05 | 0.01206 |
| HUWE1 | 4.0 | 0.980 | 1.71E−04 | 0.01460 |
| HS6ST1 | 7.5 | 0.977 | 4.86E−05 | 0.01206 |
| SOX4 | 4.0 | 0.975 | 6.76E−05 | 0.01206 |
| ZNF496 | 1.0 | 0.971 | 2.15E−05 | 0.01136 |
| CTSG | 208.0 | −0.970 | 1.28E−04 | 0.01426 |
| USP11 | 8.5 | 0.987 | 1.33E−04 | 0.01450 |
| PCOLCE2 | 219.5 | −0.962 | 3.12E−04 | 0.01723 |
| SET | 1.0 | 0.958 | 1.66E−04 | 0.01460 |
| BCAT1 | 18.5 | 0.954 | 1.82E−04 | 0.01460 |
| WDR43 | 21.5 | 0.954 | 3.27E−03 | 0.04671 |

TABLE 4

| Gene Name | Statistic | Median | p. value | q. value |
|---|---|---|---|---|
| FOXM1 | 61 | −6.44 | 6.58E−03 | 0.02696 |
| Tap78a_(tetramer)_(complex) | 73 | −2.90 | 7.94E−03 | 0.03088 |
| SPI1 | 56 | −2.69 | 4.34E−03 | 0.01998 |
| APOBEC3G_(family) | 45 | −2.58 | 2.63E−03 | 0.01474 |
| FOXA2 | 26 | −2.33 | 3.19E−05 | 0.00197 |
| HIF2A/ARNT_(complex) | 51 | −2.21 | 9.35E−04 | 0.00730 |
| p-T611_S730_S789- | 21 | −1.98 | 1.26E−04 | 0.00280 |
| IL23/IL23R/JAK2/ | 47 | −1.95 | 1.97E−03 | 0.01153 |
| TYK2_(complex) | | | | |
| E2F4/DP2/p107- | 57 | −1.86 | 4.72E−03 | 0.02133 |
| p130_(complex) | | | | |
| STAT6_(dimer)_(complex) | 62 | −1.86 | 7.13E−03 | 0.02855 |
| TRAF3 | 78 | −1.82 | 1.37E−02 | 0.04550 |
| TBC1D4 | 55 | −1.79 | 6.93E−03 | 0.02797 |
| Myb/CYP-40_(complex) | 21 | −1.75 | 3.92E−04 | 0.00445 |
| TCF4/beta_catenin_(complex) | 64 | −1.74 | 1.46E−02 | 0.04764 |
| SHMT1 | 6 | −1.72 | 8.80E−04 | 0.00713 |
| BAD | 55 | −1.72 | 2.30E−03 | 0.01286 |
| HELLS | 28 | −1.72 | 2.49E−03 | 0.01342 |
| CHST1 | 0 | −1.71 | 6.51E−04 | 0.00589 |
| CNR1 | 0 | −1.71 | 1.08E−03 | 0.00789 |
| CACNA1E | 0 | −1.71 | 8.37E−04 | 0.00694 |

As can be readily taken from the data and Tables 2-4 above, significant differences in gene expression and changes in inferred pathway activity were discovered. As such the changed genes could be employed in a model to differentiate between MDS and AML, and/or to predict progression time and/or likelihood of progression. Moreover, the inventors noted that selected genes with high differential expression and/or differences in inferred pathway activity were transcription factors or closely related to transcription factors and/or targets of these factors. Therefore, in at least some aspects of the inventive subject matter, the inventors contemplate use of these genes and/or targets of these factors in a diagnostic and/or predictive model for MDS/AML transition.

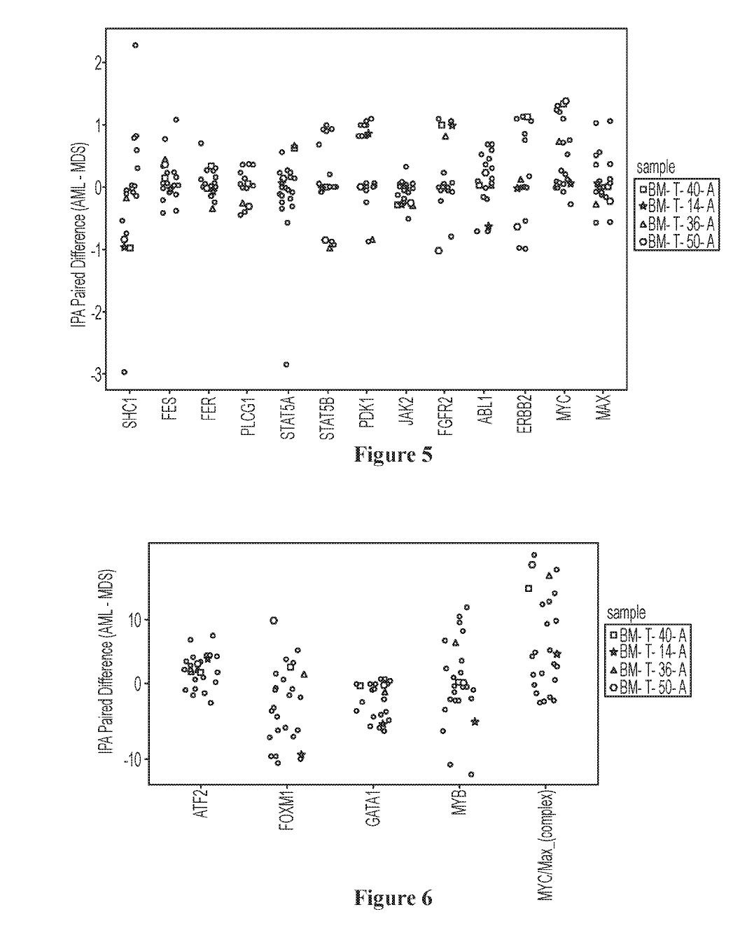

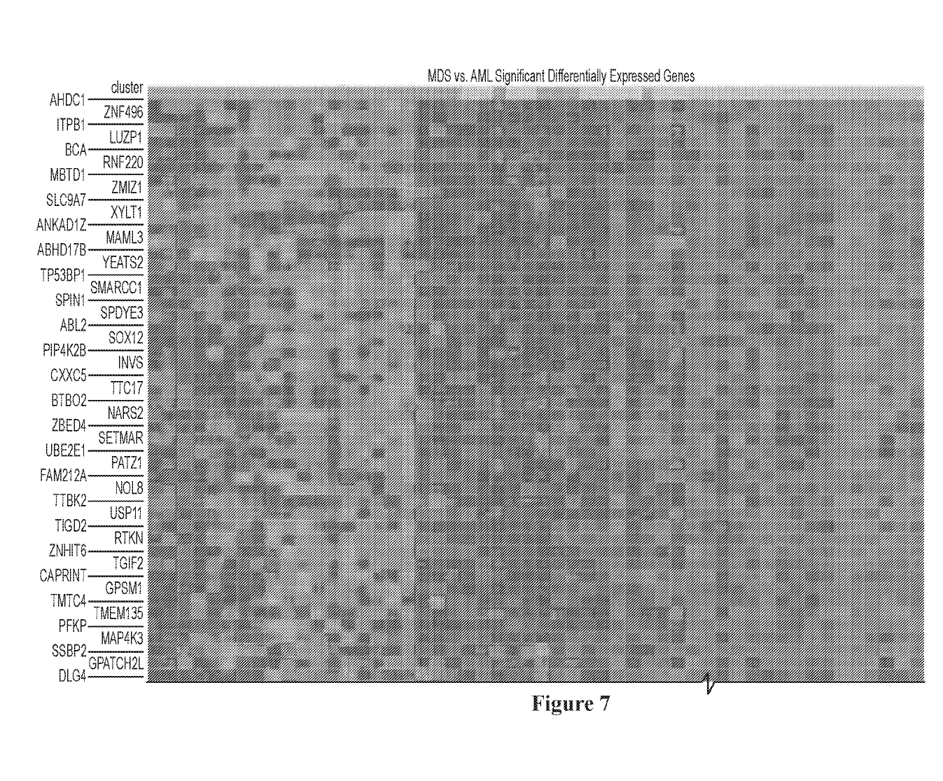

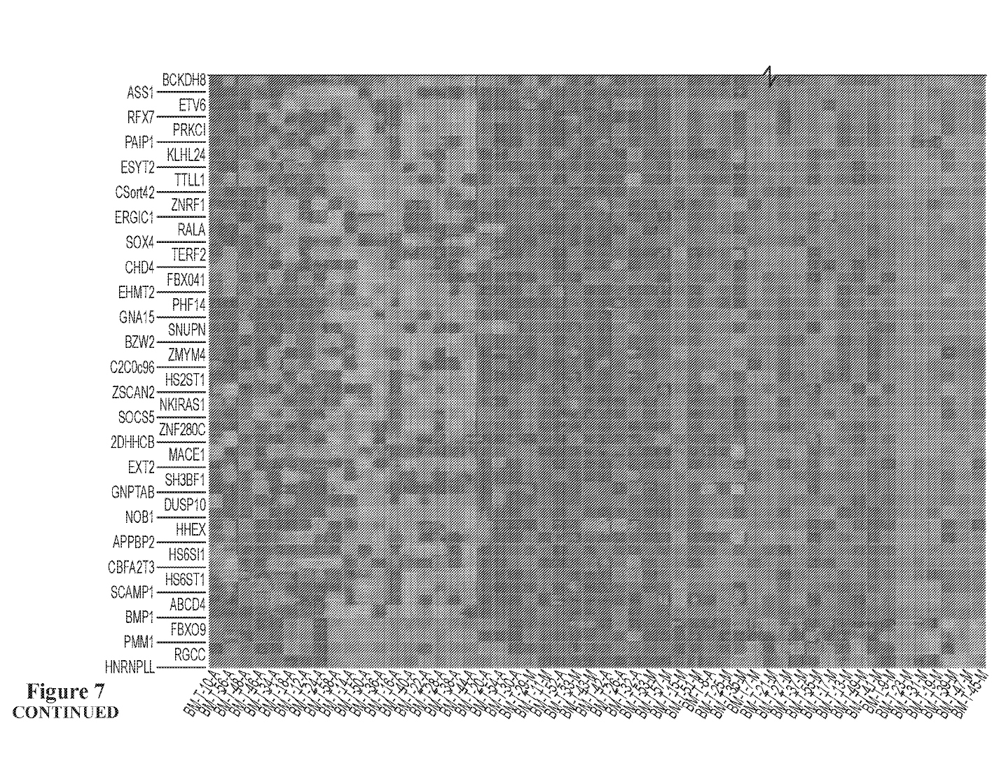

FIG. 4 is a graph exemplarily depicting the fold-change in gene expression of selected genes in AML versus MDS, and FIGS. 5-6 are graphs depicting exemplary paired differences of inferred pathway activities between AML and MDS for selected genes. Based on the notable expression differences between AML and MDS, the inventors investigated whether certain genes could be used in a quantitative and predictive model, and FIG. 7 is an exemplary heat map for 95 differentially expressed genes having statistically significant differences in gene expression. Here, the expression between AML and MDS was compared using t-tests and shown to have an alpha value of 0.05, Bonferroni corrected for testing >19K hypotheses. Of course, it should be appreciated that the statistical cut-off and particular method of comparison may be changed. Thus, and all alternative methods are deemed suitable for use herein. In another calculation, the inventors then used the 95 differentially expressed genes for building progression predictors.

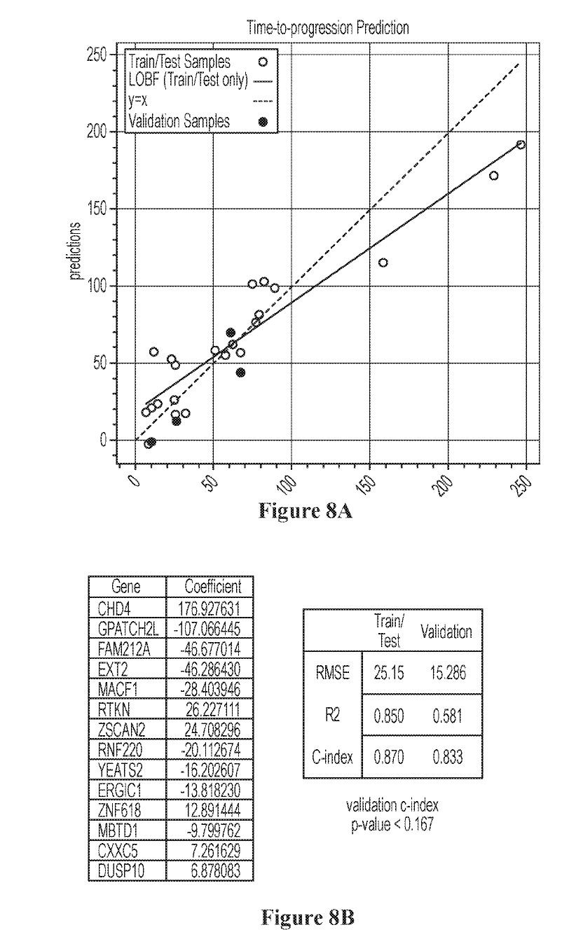

More specifically, in one example, 4/26 samples were held out for validation. Three normalizations were compared and ten regression algorithms were tested in a 6-fold cross-validation. As is shown in FIG. 8, raw expression data with Lasso least angle regression (LassoLARS) performed best in testing samples (average RMSE=65.04, average concordance index was 0.58). Interestingly, the Lassos reduced the features from the initial 95 to 14, which renders predictive and quantitative analysis relatively simple. As can be seen from FIG. 8A, a fully trained regression function can be built that quantitatively predicts from the expression values of genes listed in FIG. 8B.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

1. A method of predicting time of progression from MDS to AML in a patient, comprising:
quantifying expression of a plurality of genes of a sample of the patient containing myelodysplastic cells;
wherein the plurality of genes have an above-average difference between MDS and AML with respect to at least one of mRNA expression and inferred pathway activity; and
using the plurality of genes having the above-average difference between MDS and AML in a prediction model to calculate a likely time of progression from MDS to AML.

2. The method of claim 1 wherein the plurality of genes have an above-average difference between MDS and AML with respect to mRNA expression.

3. The method of claim 1 wherein the plurality of genes have an above-average difference between MDS and AML with respect to inferred pathway activity.

4. The method of claim 1 wherein the plurality of genes are selected from the group consisting of CHD4, GPATCH2L, FAM212A, EXT2, MACF1, RTKN, ZSCAN2, RNF220, YEATS2, ERGIC1, ZNF618, MBTD1, CXXC5, and DUSP10.

5. The method of claim 1 wherein the prediction model is based on a plurality of differentially expressed genes in which at least 50 genes are differentially expressed as determined by t-test and an alpha of 0.05.

6. The method of claim 5 wherein the plurality of differentially expressed genes are selected from the group consisting of differentially expressed genes of FIG. 7.

7. The method of claim 1 wherein the prediction model is built using a regression algorithm.

8. The method of claim 7 wherein the regression algorithm is a lasso least-angle regression.

9. The method of claim 1 wherein the prediction model provides predictions up to at least 120 months.

10. The method of claim 1 wherein the step of quantifying expression of the plurality of genes uses whole transcriptome RNAseq data.

11. The method of claim 10 further comprising a step of identifying a druggable target in the whole transcriptome RNAseq data.

12. The method of claim 1 further comprising a step of generating or updating a report with a treatment recommendation.

13. A method of generating a model for predicting time for MDS to AML transition, comprising:
quantifying expression of a plurality of genes of a sample containing MDS cells;
quantifying expression of a plurality of genes of a sample containing AML cells;
optionally calculating inferred pathway activities for the plurality of genes of the sample containing MDS cells and the plurality of genes of the sample containing AML cells;
identifying a plurality of genes with an above-average difference between the MDS cells and the AML cells with respect to at least one of mRNA expression and inferred pathway activity; and
using the plurality of genes with the above-average difference between the MDS cells and the AML cells to build a prediction model that calculates a likely time of progression from MDS to AML.

14. The method of claim 13 wherein the plurality of genes have an above-average difference between MDS and AML with respect to mRNA expression.

15. The method of claim 13 wherein the plurality of genes have an above-average difference between MDS and AML with respect to inferred pathway activity.

16. The method of claim 13 wherein the prediction model is based on a plurality of differentially expressed genes in which at least 50 genes are differentially expressed as determined by t-test and an alpha of 0.05.

17. The method of claim 13 wherein the plurality of genes with the above-average difference between the MDS cells and the AML cells are selected from the group consisting of CHD4, GPATCH2L, FAM212A, EXT2, MACF1, RTKN, ZSCAN2, RNF220, YEATS2, ERGIC1, ZNF618, MBTD1, CXXC5, and DUSP10.

18. The method of claim 13 wherein the prediction model is built using a regression algorithm.

19. The method of claim 18 wherein the regression algorithm is a lasso least-angle regression.

20. The method of claim 19 wherein the steps of quantifying expression use whole transcriptome RNAseq data.
